(19) United States
(12) Patent Application Publication
(10) Pub. No.: US 2025/0295854 A1
(43) Pub. Date: Sep. 25, 2025

(54) ACCURACY OF BASAL DELIVERY AMOUNTS

(71) Applicant: INSULET CORPORATION, Acton, MA (US)

(72) Inventors: Joon Bok LEE, Acton, MA (US); Yibin ZHENG, Hartland, WI (US); William WHITELEY, Knoxville, TN (US); Jason O’CONNOR, Acton, MA (US)

(21) Appl. No.: 19/083,776

(22) Filed: Mar. 19, 2025

Related U.S. Application Data
(60) Provisional application No. 63/567,045, filed on Mar. 19, 2024.

(51) Int. Cl. A61M 5/172

(52) U.S. Cl. CPC A61M 5/1723; A61M 2230/201

(57) ABSTRACT

Exemplary embodiments may attempt to account for more substantial deviations in a user's basal insulin needs and reduce or eliminate glucose level excursions that may result from such deviations more quickly than conventional insulin pump systems. The exemplary embodiments may rely upon a user's recent basal insulin delivery history to establish a current estimate of the user's basal insulin needs. Exemplary embodiments may calculate a reliability metric for a TDI value that reflects that degree of variance in TDI value for a user over a recent period, such as over multiple days or weeks. The reliability indicator may be used to adjust the TDI value that is used to establish a user's daily basal insulin needs and hourly basal insulin needs. In determining a user's basal insulin needs, the exemplary embodiments may not only look to recent basal insulin deliveries but also may account for correction boluses and/or meal boluses.

### CROSS-REFERENCE TO RELATED APPLICATIONS

This application claims priority to and the benefit of U.S. Provisional Application No. 63/567,045, filed Mar. 19, 2024, the entirety of which is incorporated herein by reference.

### BACKGROUND

Conventional insulin pump systems may provide basal insulin deliveries to users. The conventional insulin pump systems may include control software that controls operation of the insulin pump systems. Part of the control of the operation provided by such control software includes determining a user's basal insulin needs on a daily and/or hourly basis. In some conventional insulin pump systems, a user's hourly basal insulin needs may be calculated as a proportion of the user's total daily insulin (TDI), such as by using the following equations:

$${b}_{i} = P \cdot \frac{TDI}{24} \qquad ( Equation 1 )$$

$$TDI = \frac{{\sum }_{j = 1}^{288 \cdot 3} I ( j )}{3} \qquad ( Equation 2 )$$

where P is a proportionality parameter, TDI is the total daily insulin average over three days, I(j) is the amount of insulin delivered either manually or delivered automatically by a delivery device during operational cycle j, and bᵢ is the user's hourly basal needs at hour i.

Conventionally, this proportionality parameter P may be calculated by assessing the proportion of manual insulin deliveries versus automated insulin deliveries, whether directly via a user's input basal profiles or through an automated insulin delivery algorithm, over a fixed period of time, as follows:

$${P}_{i} = 1 - \frac{{\sum }_{j = 1}^{288 \cdot 3} {I}_{m} ( {k}_{i} - j )}{{\sum }_{j = 1}^{288 \cdot 3} I ( {k}_{i} - j )} \qquad ( Equation 3 )$$

where Pᵢ is the proportionality parameter for hour i, I( ) is the insulin that was automatically or manually delivered to the user during a specified operational cycle over a period of 3 days, and Iₘ( ) is the quantity of manual boluses for the specified operational cycle. In this example, the equation is defined for the previous 3 days, or 72 hours, and the operational cycles are presumed to each be 5 minutes in length (hence, there are 288 cycles in day). This proportionality parameter may range from 0 to 1, based on the quantity of manual boluses versus all insulin deliveries.

### SUMMARY

In accordance with a first inventive facet, a medicament delivery system includes a non-transitory computer-readable storage storing computer programming instructions and a processor. The processor is configured for executing the computer programming instructions to cause the processor to determine a proportionality parameter for a user based on a quantity of insulin that was delivered manually to the user and that is still active over a previous period and/or an amount of insulin on board (IOB) that is still active and that was delivered as one or more meal boluses to the user over the previous period. The processor is further configured for executing the computer programming instructions to cause the processor to determine basal needs of the user for a time window as a portion of the total daily insulin (TDI) of the user determined by the proportionality parameter.

The quantity of insulin that was delivered manually to the user and that is still active over the previous period may be a quantity of manually delivered correction boluses over the previous period. The quantity of insulin that was delivered manually to the user and that is still active over the previous period may be a quantity of manually delivered meal and correction boluses over the previous period. The previous period may be 5 hours in length or another time period. The determining of the basal needs of the user for a time window may include multiplying the proportionality parameter by total daily insulin (TDI) divided by 24. The time window may be an hour in length or another time period. The proportionality parameter may be constrained to assume a value in a range extending from a minimum value to a maximum value.

In accordance with another inventive facet, a medicament delivery system includes a non-transitory computer-readable storage storing computer programming instructions and a processor. The processor is configured for executing the computer programming instructions to cause the processor to determine a reliability factor for a current total daily insulin (TDI) value for a user based on an amount that a current glucose level value of the user differs from a target glucose level for the user and based on variance of TDI values over time and to determine basal insulin needs over a time window for a user by applying the reliability factor to the current TDI.

The determining of the basal insulin needs may include multiplying the reliability factor by a tuning factor to yield a product. The determining of the basal insulin needs may include adding the product with an additional tuning factor to determine a sum and multiplying the current TDI value divided by how many time periods of a length equal to a length of the time window are in a day to determine the basal insulin needs over the time window. The determining of the reliability factor may include determining a standard deviation of the TDI values over a period of multiple days. The reliability metric may be constrained to have a value greater than zero. The time window may be an hour in duration.

In accordance with an additional inventive facet, a medicament delivery system includes a medicament delivery device for delivering insulin to a user. The medicament delivery device includes a non-transitory computer-readable storage storing computer programming instructions and a processor. The processor is configured for executing the computer programming instructions to cause the processor to sum basal insulin deliveries by the medicament delivery device to the user and correction bolus deliveries to the user over a period exceeding a day to determine a total, wherein the period contains intervals, to determine an average of the basal insulin deliveries per interval for the total, and to determine an amount of basal insulin to be delivered over a next interval based on the determined average of the basal insulin deliveries per interval.

The computer programming instructions may further cause the processor to identify basal medicament deliveries that are postprandial. In summing the basal medicament deliveries, for a non-postprandial basal medicament delivery at a cycle where a glucose reading for the cycle is above a high glucose level threshold, the insulin amount that was delivered at the cycle may be replaced with an amount equal to an input hourly basal amount of the user divided by a number of cycles in an hour. In summing the basal medicament deliveries, for a non-postprandial basal medicament delivery at a cycle where a glucose reading for the cycle is below a low glucose level threshold, the insulin amount that was delivered at the cycle may be replaced with an amount equal to an input hourly basal amount of the user divided by a number of cycles in an hour. The determined average of the basal insulin deliveries per interval may be the determined amount of basal to be delivered over the next interval. The amount of basal insulin to be delivered at the next interval may be a sum of a weighted value derived from total daily insulin (TDI) of the user and a weighted value of the determined average of the basal insulin deliveries per interval.

### BRIEF DESCRIPTION OF THE DRAWINGS

FIG. 1 depicts a block diagram of an illustrative medicament delivery system that is suitable for delivering a medicament, such as insulin, to a user in accordance with the exemplary embodiments.

FIG. 2 depicts a flowchart of illustrative steps that may be performed in exemplary embodiments to account for recently delivered insulin boluses that still have insulin action.

FIG. 3 depicts illustrative steps that may be performed in exemplary embodiments to adjust a proportionality parameter in accordance with a first option.

FIG. 4 depicts a flowchart of illustrative steps that may be performed in exemplary embodiments to calculate an adjusted proportionality parameter while accounting for the IOB resulting from manual meal bolus deliveries.

FIG. 5 depicts a flowchart 500 of illustrative steps that may be performed in exemplary embodiments to determine a proportionality parameter.

FIG. 6 depicts a flowchart of illustrative steps that may be performed in using a reliability metric to calculate the user's initial basal insulin needs.

FIG. 7 depicts a flowchart of illustrative steps that may be performed in exemplary embodiments to determine the reliability metric.

FIG. 8 depicts a flowchart of illustrative steps that may be performed in exemplary embodiments to calculate the hourly basal needs of the user using the reliability metric.

FIG. 9 depicts a flowchart of illustrative steps that may be performed in exemplary embodiments to make an adjustment to basal needs of the user to account for the total manual insulin deliveries in a recent period.

FIG. 10 depicts a flowchart of illustrative steps that may be performed in exemplary embodiments to account for some of the manual bolus deliveries in determining the user's basal insulin needs.

FIG. 11 depicts a flowchart of illustrative steps that may be performed in exemplary embodiments to determine the value for basal meal insulin deliveries that are used in determining the user's basal insulin needs.

FIG. 12 depicts a flowchart of illustrative steps that may be performed in exemplary embodiments to calculate the current basal insulin needs of the user based on the average basal insulin deliveries to the user over the previous N cycles.

FIG. 13 depicts a flowchart of illustrative steps that may be performed by exemplary embodiments to calculate the user's basal needs based on the sum of basal deliveries and correction bolus deliveries.

FIG. 14 depicts a flowchart of illustrative steps that may be performed by exemplary embodiments to account for inaccuracies in a user's non-bolus insulin needs by removing any basal deviations following user boluses that are paired with significant glucose excursions in determining a user's basal needs.

### DETAILED DESCRIPTION

The conventional insulin pump systems assume that the user's basal insulin needs will not substantially deviate from the average needs over the most recent days. Unfortunately, such may not be the case, and as a result, glucose level management by the insulin pump system may suffer when there are substantial deviations. The user may have substantial glucose level excursions in such instances and it may take an extended period of time for the insulin pump systems to compensate for such glucose level excursions.

Exemplary embodiments may attempt to account for more substantial deviations in a user's basal insulin needs and reduce or eliminate glucose level excursions that may result from such deviations more quickly than conventional insulin pump systems. The exemplary embodiments may rely upon a user's recent basal insulin delivery history to establish a current estimate of the user's basal insulin needs. In some exemplary embodiments, the proportionality parameter P that specifies what portion of TDI is designated for basal insulin deliveries may be adjusted based on recent manual insulin deliveries that have been delivered in a time window of a duration of insulin action (DIA), which may be, for example, 5 or 6 hours. In other exemplary embodiments, the proportionality parameter may be adjusted based upon the insulin on board (IOB) of a user.

Exemplary embodiments may calculate a reliability metric for a TDI value that reflects that degree of variance in the TDI value for a user over a recent period, such as over multiple days or weeks. The reliability metric may also reflect how much glucose level values of the user deviate relative from a target glucose level over the period to reflect how well glucose level of the user is being managed. The reliability indicator may be used to adjust the TDI value that is used to establish a user's daily basal insulin needs and hourly basal insulin needs. As a result, TDI values that deviate from a recent norm and TDI values from a period where glucose level management has been poor are assigned lower reliability and may be weighted less in adjusting the basal insulin needs of the user.

In determining a user's basal insulin needs, the exemplary embodiments may not only look to recent basal insulin deliveries but also may account for correction boluses and/or meal boluses. In addition, the determination of a user's basal insulin needs may identify basal insulin deliveries that are in a post-prandial period and may treat those basal insulin deliveries differently than basal insulin deliveries that are not in a post-prandial period. Specifically, where there are glucose excursions within a post-prandial period, the basal insulin deliveries are replaced by the baseline basal insulin delivery values.

Exemplary embodiments may adjust the user's basal insulin needs in real time, such as in each operational cycle. The user's average basal insulin needs over a period of N days may be used to establish the user's current basal insulin needs. The history may be augmented to include both basal insulin deliveries as well as correction bolus histories.

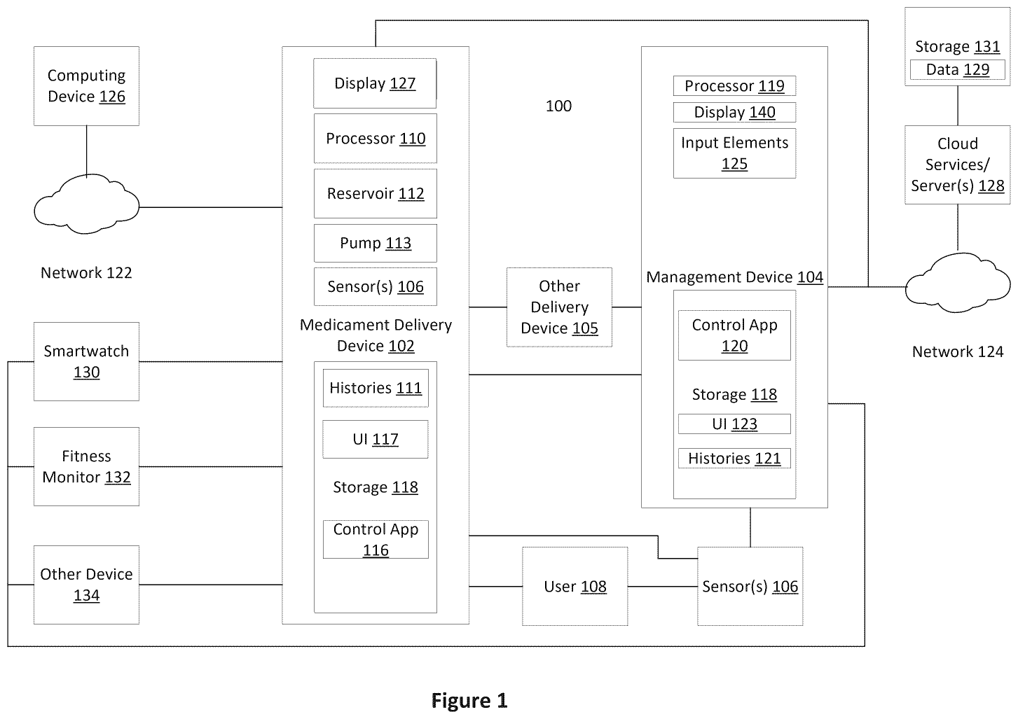

FIG. 1 depicts a block diagram of an illustrative medicament delivery system 100 that is suitable for delivering a medicament, such as insulin, to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no external tubing and an infusion location directly under the medicament delivery device 102, or may be a device carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal deliveries, a history of bolus deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when the display is a touchscreen. The management device 104 may further include input elements 125, such as a keyboard, button, knobs, or the like, for receiving input of the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen (such as an insulin pen), may be accounted for (e.g., in determining insulin on board (IOB)) or may be provided for also delivering medicament to the user 108.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may one medicament, such as insulin, for lowering glucose levels of the user 108 and also deliver another medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. In other embodiments, the medicament delivery device 102 may deliver concentrated insulin. In some embodiments, the medicament or medicament delivered by the medicament delivery device may be a coformulation of two or more of those medicaments identified above. In a preferred embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to be to offset the expected rise in glucose level of the user 108 from ingesting a meal or for correcting a persistently elevated glucose level (i.e., one that is persistently higher than a target glucose level). Boluses tend to be one time deliveries for offsetting a meal or for correcting a glucose level and tend to be larger than bolus insulin deliveries. Insulin boluses may be delivered manually by the user 108, such as via a syringe, or may, in some exemplary embodiments, be delivered by the medicament delivery device 102. Basal insulin doses tend to be smaller than insulin bolus doses and are delivered periodically, such as once each operational cycle of the control approach of the medicament delivery device 102 (e.g., every 5 minutes). The aim of the basal insulin deliveries is to keep the user's glucose level within a target range that is desirable using small ongoing insulin doses.

One of the problems with the conventional approach of determining hourly basal delivery amounts from historic TDI of the user is that manual deliveries may skew the hourly basal delivery amounts. Specifically, it is problematic when the user's actual manual basal deliveries significantly deviate from baseline assumptions regarding manual deliveries that are based on manual delivery history. For instance, when a user manually delivers a bolus of medicament and has not delivered a bolus for a long period, this may increase the user's risk of hypoglycemia due to the basal delivery amount being elevated since the user has not delivered a manual bolus in a long time. Exemplary embodiments may account for such deviations in manual deliveries to produce a better hourly delivery amount that more accurately reflects the true basal insulin needs of the user 108.

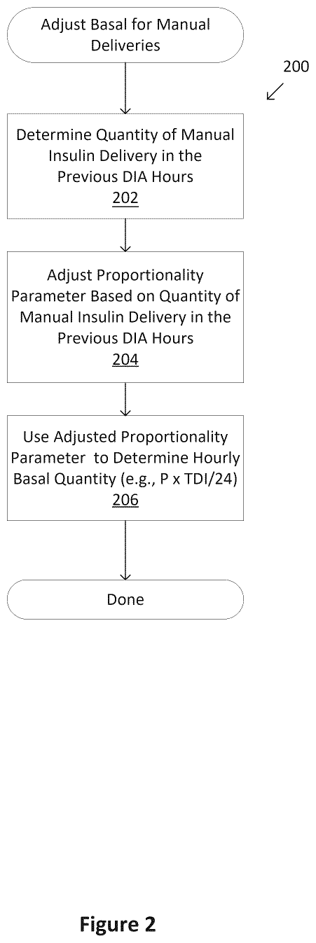

The exemplary embodiments may provide several methods to modulate the assumptions of the user's basal insulin needs by the control application of the medicament delivery device when the user's manual insulin delivery patterns deviate significantly from the baseline assumptions. To produce a better estimated of an hourly basal insulin amount, the exemplary embodiments may account for manual insulin doses that were delivered recently enough to still have insulin action by changing the proportionality parameter that determines the amount of TDI that is presumed to be for basal insulin deliveries. FIG. 2 depicts a flowchart 200 of illustrative steps that may be performed in exemplary embodiments to account for such recently delivered insulin boluses that still have insulin action. At 202, a quantity of manual insulin delivery in the previous hours that constitute a duration of insulin action (DIA) may be determined. This aggregates the quantity of any manual insulin deliveries that still have any insulin action, or insulin that has not yet acted in the body. The DIA may be established as a fixed value, such as 5 or 6 hours. Other values may be chosen for the DIA variable. This quantity may be calculated as:

$${\sum }_{j = 1}^{DIA \cdot 12} {I}_{m} ( j ) \qquad ( Equation 4 )$$

where DIA is the duration of insulin action specified in hours, Iₘ(j) is the quantity of insulin delivered manually during cycle j, j is a cycle index where a cycle is presumed to be 5 minutes in length, and TDI is total daily insulin.

At 204, the adjusted proportionality parameter, designated as Pₐ(k), may be adjusted based on the quantity of manual insulin delivery to the user in the previous DIA hours. Illustrative ways for adjusting the proportionality parameter in exemplary embodiments are discussed in more detail below. At 206, the adjusted proportionality parameter is used to determine the hourly basal quantity for the next hour. For example, as in Equation 1, the proportionality parameter may be multiplied by TDI/24 to determine the hourly basal quantity.

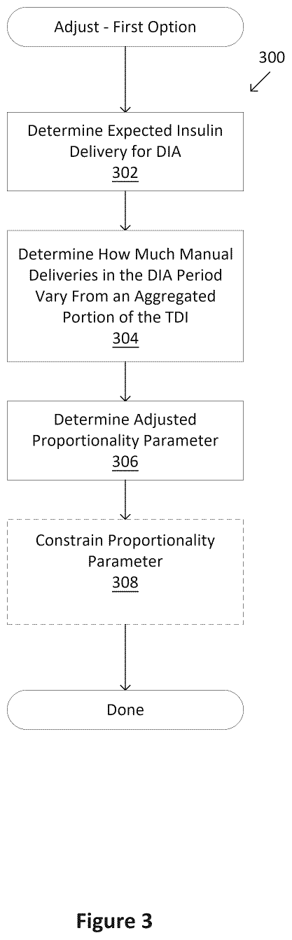

One way to adjust the proportionality parameter in exemplary embodiments is set forth in the flowchart 300 of FIG. 3. The flowchart 300 depicts illustrative steps that may be performed in exemplary embodiments to adjust the proportionality parameter in accordance with a first option. At 302, an expected amount of insulin to be delivered to the user during the DIA period is determined. For example, the product of TDI/24 and DIA may be calculated. The fraction TDI/24 specifies an hourly average of TDI. Multiplying that fraction by DIA results in a quantity of insulin equal to the hourly average of TDI aggregated over the DIA period (e.g., 5 or 6 hours). At 304, how much the actual manual deliveries in the DIA period vary from the quantity assumed in the TDI value may be determined. For example, the sum of manual insulin deliveries over the past DIA hours may be divided by the product calculated at 302. The resulting value represents a ratio reflecting how much the actual manual deliveries in the DIA period vary from the quantity assumed in the TDI value. At 306, the adjusted proportionality parameter may be determined. For example, 1 minus the resulting value from 304 may be used as the adjusted proportionality parameter. Hence, in this particular example, the adjusted proportionality parameter may be expressed as follows:

$${P}_{a} ( k ) = 1 - \frac{{\sum }_{j = 1}^{DIA \cdot 12} {I}_{m} ( k - j )}{\frac{TDI}{24} \cdot DIA} . \qquad ( Equation 5 )$$

where k is a cycle index that identifies a cycle number and cycles are 5 minutes in length.

In some exemplary embodiments, at 308, the adjusted proportionality parameter may be constrained. For example, the adjusted proportionality parameter may be constrained to have a value between a minimum Pₘᵢₙ and a maximum Pₘₐₓ. Since this step is optional for some embodiments, the box for this step is shown in phantom form. The minimum Pₘᵢₙ may be, for example, 0, 0.25, or 0.40. The maximum Pₘₐₓ may be, for example, 1, 0.75, or 0.90. It will be appreciated that other values in the range between 0 and 1 may be chosen for the minimum and maximum. The minimum and maximum values help to ensure that more extreme values that diverge from the 0.5 ideal split proportionality parameter value are not used.

When the minimum and maximum are used, the final adjusted proportionality parameter may be calculated in this example as follows:

$${P}_{a} ( k ) = max ( {P}_{min} , min ( {P}_{max} , 1 - \frac{{\sum }_{j = 1}^{DIA \cdot 12} {I}_{m} ( k - j )}{\frac{TDI}{24} \cdot DIA} ) ) \qquad ( Equation 6 )$$

where the max( ) function chooses the maximum among a set of values and the min( ) function chooses the minimum among a set of values.

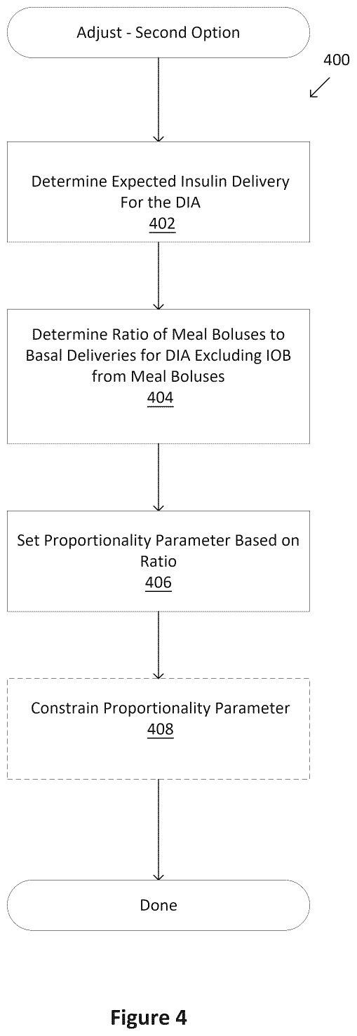

In some exemplary embodiments, the manual insulin deliveries in the form of meal boluses may be taken into account in determining the adjusted hourly basal delivery amount by accounting for the insulin on board (IOB) resulting from such manual insulin deliveries. As explained below, the insulin that has not yet acted may be subtracted out in determining the proportionality parameter. FIG. 4 depicts a flowchart of illustrative steps that may be performed in exemplary embodiments to calculate the adjusted proportionality parameter while accounting for the IOB resulting from manual meal bolus deliveries. At 402, an expected amount of insulin to be delivered to the user during the DIA period based on TDI is determined, as discussed above relative to step 302. At 404, the ratio of meal boluses to basal deliveries for the DIA period excluding the IOB remaining from the meal boluses may be determined. For example, the sum of the meal boluses minus the IOB that remains from the meal cycles is determined (e.g., Σⱼ₌₁^{DIA·12}Iₘ (k−j)−IOBₘₑₐₗ(k)) and divided by the expected total insulin deliveries for the DIA period (e.g., TDI/24×24) may be calculated. At 406, the adjusted proportionality parameter may be set, for example, as:

$${P}_{a} ( k ) = 1 - \frac{{\sum }_{j = 1}^{DIA \cdot 12} {I}_{m} ( k - j ) - {IOB}_{meal} ( k )}{\frac{TDI}{24} \cdot DIA} \qquad ( Equation 7 )$$

Optionally, at 408, the proportionality parameter value may be constrained. For example, the proportionality parameter may be constrained to a range between a minimum Pₘᵢₙ and a maximum Pₘₐₓ such as discussed above relative to step 308. In that case, a suitable equation for determining the adjusted proportionality parameter is:

$${P}_{a} ( k ) = max ( {P}_{min} , min ( {P}_{max} , 1 - \frac{{\sum }_{j = 1}^{DIA \cdot 12} {I}_{m} ( k - j ) - {IOB}_{meal} ( k )}{\frac{TDI}{24} \cdot DIA} ) ) \qquad ( Equation 8 )$$

where IOBₘₑₐₗ(k) may be calculated as:

$${IOB}_{meal} ( k ) = [ {I}_{meal} ( k - 1 ) {I}_{meal} ( k - 2 ) … … {I}_{meal} ( k - DIA \cdot 12 - 1 ) {I}_{meal} ( i - DIA \cdot 12 ) ] \cdot {D}_{DIA} \qquad ( Equation 9 )$$

where Iₘₑₐₗ( ) is the amount of meal bolus insulin that was manually delivered during the specified cycle and D_{DIA} is a vector representing the hourly decay curve for the user 108. D_{DIA} may be expressed in matrix form as:

$${D}_{DIA} = [ \begin{matrix}1 \\ 1 - \frac{1}{DIA \cdot 12} \\ : \\ 1 - \frac{DIA \cdot 12 - 1}{DIA \cdot 12} \\ 0\end{matrix} ] \qquad ( Equaton 10 )$$

It should be appreciated that the adjustments to the proportionality parameters described above relative to FIGS. 3 and 4 may be incorporated by non-linear functions, such as quadratic functions or square root functions, in some exemplary embodiments to reduce the impact of large user boluses from incurring step changes in the proportion of boluses.

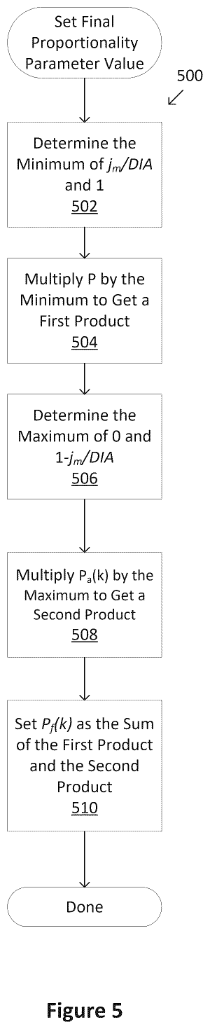

The final proportionality parameter may be determined using the adjusted proportionality parameter value. In exemplary embodiments, the final proportionality parameter P_{f}(k) may be calculated as a moving average of the system's typical assumptions for proportionality P and the adjusted proportionality parameter. For example, P_{f}(k) may be calculated as follows:

$${P}_{f} ( k ) = min ( \frac{{j}_{m}}{DIA} , 1 ) P + max ( 0 , 1 - \frac{{j}_{m}}{DIA} ) {P}_{a} ( k ) \qquad ( Equation 11 )$$

where jₘ is the number of cycles since the last manual bolus of insulin was delivered and DIA is expressed in cycles. FIG. 5 depicts a flowchart 500 of illustrative steps that may be performed in exemplary embodiments to determine P_{f}(k) per this equation. At 502, the minimum of

$$\frac{{j}_{m}}{DIA}$$

and 1 may be determined. Choosing the minimum prevents the value from exceeding 1. At 504, the system's typical assumption of proportionality for the user (i.e., P) may be multiplied by the determined minimum to get a first product. The ratio

$$\frac{{j}_{m}}{DIA}$$

scales P based on the age of the last may be determined to manually delivered insulin bolus. At 506, the maximum of 0 and

$$1 - \frac{{j}_{m}}{DIA}$$

may be determined to ensure that weight applied to Pₐ(k) is not negative. At 508, the resulting maximum (the weight) may be multiplied with Pₐ(k) to get a second product. At 510, P_{f}(k) may be set as the sum of the first product and the second product.

As was mentioned above, the manual insulin deliveries of a user may vary over time and thus may cause the TDI of the user 108 to vary as well. Since the medicament delivery device 102 may rely upon TDI to determine basal delivery amounts, it is desirable to better appreciate how reliable a TDI value is and to be able to compensate for a lack of reliability. Some exemplary embodiments calculate a reliability metric and use the reliability metric in calculating the user's initial basal needs.

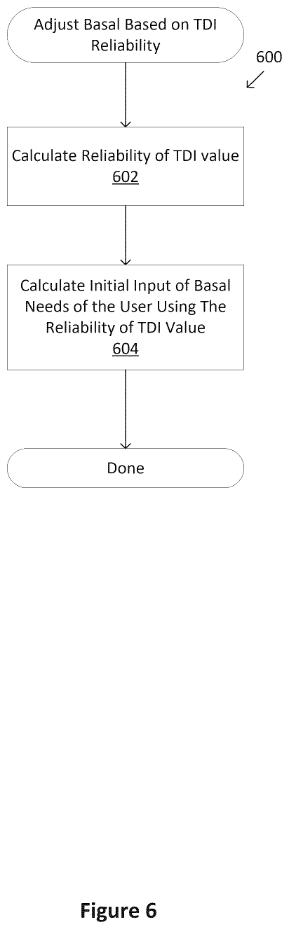

FIG. 6 depicts a flowchart 600 of illustrative steps that may be performed in using the reliability metric to calculate the user's initial basal insulin needs. At 602, the reliability metric for the TDI value to be used in establishing a user's basal insulin needs is determined. One approach to calculating the reliability metric is set forth below. That said, other reliability metrics may be used. At 604, the reliability metric is used in calculating the initial basal insulin needs of the user.

The reliability metric may be calculated in exemplary embodiments using the following equation:

$${R}_{TDI} = max ( 1 - ( \frac{STD ( {TDI}_{i - 1 , i - 2 , … i - n} )}{0.25 \cdot {TDI}_{i - 1}} ) - \frac{\vert {\overline{CGM}}_{i - 1} - 140 \vert}{48} , 0 ) \qquad ( Equation 12 )$$

where R_{TDI} is the reliability metric, i is a day index, and CGM is the magnitude of a reading from a glucose sensor, such as from a CGM.

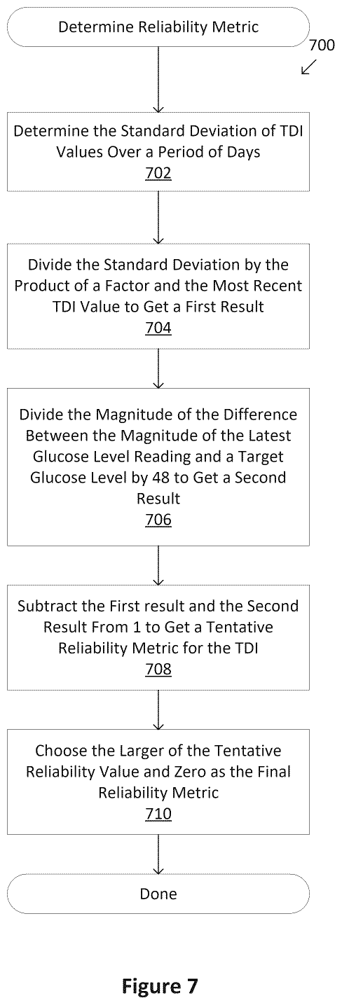

FIG. 7 depicts a flowchart of illustrative steps that may be performed in exemplary embodiments to determine the reliability metric per this equation. At 702, the standard deviation of recent TDI values may be determined (i.e., the standard deviation of the TDI values for the n most recent days, where n is a positive integer). The standard deviation gives a measure of the variance of the TDI value. Greater variance is equated with less reliability in this context. At 704, the standard deviation may be divided by the product of 0.25 and the TDI value for the previous day (i.e., TDIᵢ₋₁) to get a first result. This approach assumes that up to 25% variance is expected but greater than 25% reduces reliability. The 25% threshold is a tunable parameter. At 706, the magnitude of the difference between the magnitude of the latest glucose level reading for the user 108 and a target glucose level (assumed to be 140 in this instance but that value is customizable) is divided by 48

$$( i . e . , \frac{\vert {\overline{CGM}}_{i - 1} - 140 \vert}{48} )$$

to get a second result. Step 706 calculates any deviations from the target glucose level over a 4 hour period (that is 48 cycles). In other words, step 706 calculates

$$\frac{\vert {\overline{CGM}}_{i - 1} - 140 \vert}{48} .$$

The value 48 is customizable. At 708, the first result and the second result are subtracted from 1 to get a tentative reliability metric value. However, the reliability metric is intended to be in a range between 0 and 1, so at 710, the maximum of the tentative reliability metric and 0 is calculated and is used as the final reliability metric.

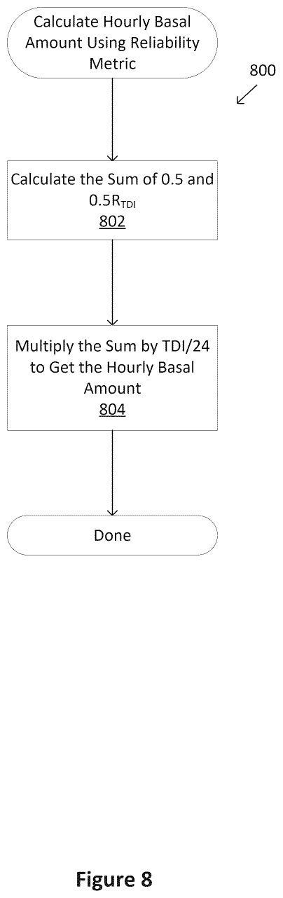

FIG. 8 depicts a flowchart 800 of illustrative steps that may be performed in exemplary embodiments to calculate the basal needs of the user 108 using the reliability metric. At 802, the sum of 0.5 and 0.5 R_{TDI} may be calculated. If R_{TDI} is 1, the sum is 1, and if R_{TDI} is 0, the sum is 0.5. At 804, the sum may be multiplied by TDI/24 to get the initial hourly basal needs of the user 108. It should be appreciated that 0.5 is a tunable value as is the weight value of 0.5. For instance, the sum could be 0.4 and 0.6 R_{TDI} or 0.6 and 0.4 R_{TDI} in some exemplary embodiments.

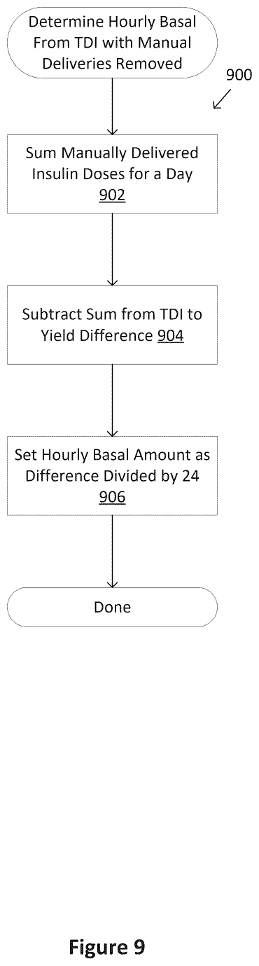

The basal needs of the user as determined at 804 may be adjusted to account for the total manual insulin deliveries in a recent period, such as in the past 24 hours. FIG. 9 depicts a flowchart 900 of illustrative steps that may be performed in exemplary embodiments to make such an adjustment. At 902, the manually delivered doses for the time period (e.g., a day) may be summed. At 904, the sum may be subtracted from TDI to yield a difference. At 906, the difference is divided by 24 to get the hourly basal needs of the user 108. This calculation may be expressed as:

$${b}_{f , i} ( k ) = \frac{TDI - {\sum }_{j = 1}^{288} {I}_{m} ( k - j )}{24} \qquad ( Equation 13 )$$

where k and j are cycle indices.

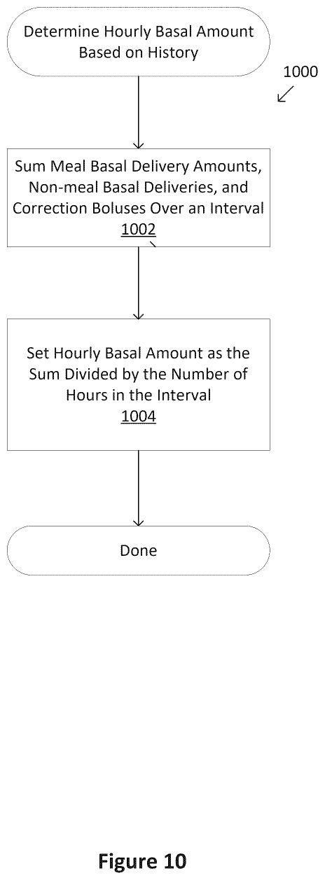

In some exemplary embodiments, the user's basal insulin needs are calculated without considering manual insulin deliveries as found with the conventional approaches that rely on TDI to calculate the user's basal insulin needs. FIG. 10 depicts a flowchart 1100 of illustrative steps that may be performed in exemplary embodiments to account for some of the manual bolus deliveries. At 1002, the meal basal delivery amounts, the non-meal basal delivery amounts and the correction boluses over an interval may be summed. The correction boluses are summed as they are viewed as a portion of the insulin needs that ideally should be handled by the basal insulin deliveries. The meal basal deliveries may be those that are delivered in a post-prandial period, such as within 5 hours of the user 108 ingesting a meal. These are treated uniquely as described below to replace basal values that resulted in poor glucose level management with basal insulin doses that are more ideal. The non-meal basal deliveries are those that are delivered outside of the post-prandial period. At 1004, the sum is divided by the number of hours in the interval to yield an hourly basal amount.

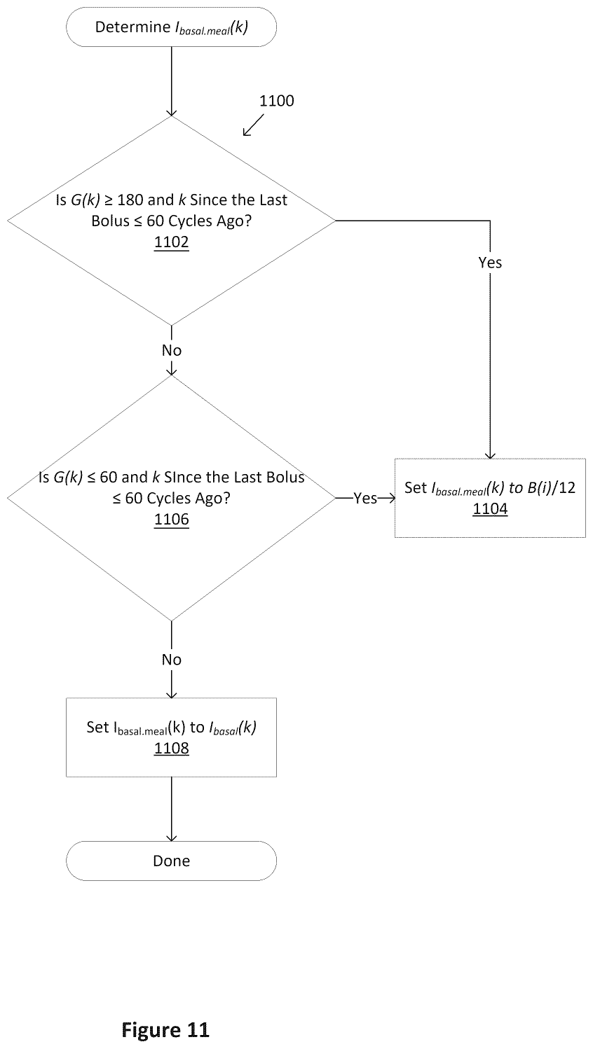

The meal basal deliveries may be given values in the calculations as follows:

$${l}_{basal , meal} ( k ) = \{ \begin{matrix}B ( i ) / 12 & G ( k ) \geq 180 and {k}_{since last bolus} \leq 60 \\ B ( i ) / 12 & G ( k ) \leq 80 and {k}_{since last bolus} \leq 60 \\ {I}_{basal} ( k ) & otherwise\end{matrix} \qquad ( Equation 14 )$$

where G(k) is the glucose level reading for the user for cycle k and B(i) is the input standard basal delivery amount for hour i. FIG. 11 depicts a flowchart 1100 of illustrative steps that may be performed in exemplary embodiments to determine the value for basal meal insulin deliveries that are used in equation 14. At 1102, a check is made whether the basal insulin delivery was made within 5 hours (i.e., 60 cycles) of a meal being ingested by the user 108 and if the user's glucose level reading for cycle k is above 180 mg/dL. If not, at 1106, a check is made whether the basal insulin delivery was made within 5 hours of a meal being ingested by the user 108 and if the user's glucose level reading for cycle k is below 80 mg/dL. At 1104, if the user 108 has a glucose level above 180 mg/dL, which is slightly hyperglycemic, or below 80 mg/dL, which is slightly hypoglycemic, and a meal was ingested within the past 5 hours by the user 108, the history values are not used; rather the input basal rate for the hour i divided by 12 (since there are 12 cycle in an hour) is used instead. The values 80 and 180 are tunable. Otherwise, at 1108, the basal delivery amount for cycle k is used.

In exemplary embodiments, the current basal insulin needs of the user may be set based on the average basal insulin deliveries to the user over the previous N cycles. One approach to determining the current basal insulin needs of the user using this approach is captured by the following equation which combines the recent basal delivery history with the baseline assumption of what basal delivery should be:

$$B ( k ) = max ( 0 , 1 - \frac{0.2}{288} {N}_{hist} ) \frac{TDI}{48} + min ( 1 , \frac{0.2}{288} {N}_{hist} ) \frac{{\sum }_{j = 1}^{{N}_{hist}} {I}_{b a s a l} ( k - j )}{\frac{{N}_{hist}}{288} / 24} \qquad ( Equation 15 )$$

where Nₕᵢₛₜ is the number of cycles in the history determined as:

$${N}_{hist} = min ( k , 288 \cdot {D}_{max} ) . \qquad ( Equation 16 )$$

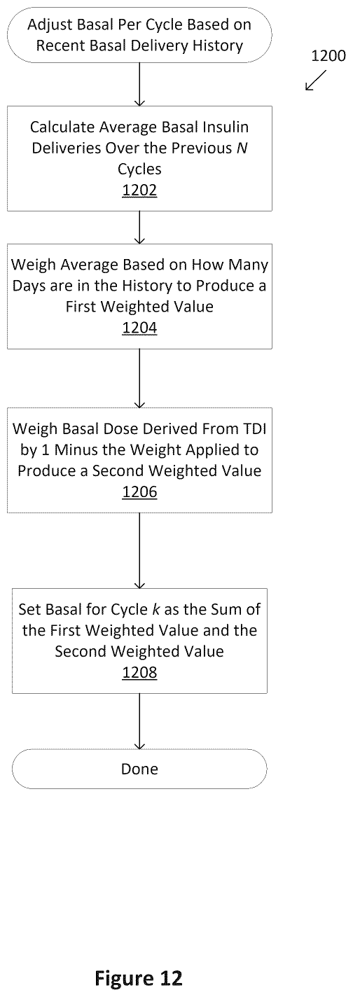

FIG. 12 depicts a flowchart 1200 of illustrative steps that may be performed in exemplary embodiments to calculate the current basal insulin needs of the user per equation 15. At 1202, the average basal insulin deliveries for the past Nₕᵢₛₜ cycles is determined. The average is determined as

$$\frac{{\sum }_{j = 1}^{{N}_{h i s t}} {I}_{b a s a l} ( k - j )}{\frac{{N}_{hist}}{288} / 24} .$$

At 1204, the average is weighed by how many cycles are in the history Nₕᵢₛₜ

$$( where the weight is min ( 1 , \frac{0.2}{288} {N}_{hist} ) ) .$$

As can be seen from Equation 16, Nₕᵢₛₜ is the smaller of the cycle index k and the product of the number of cycles in a day 288 (assuming 5 minute cycles) and the maximum number of days in the history (i.e., Dₘₐₓ, which may be, for example, 3 days). At 1206, the user basal dose per hour (i.e., TDI/48) is weighed by the max of

$$( 0 , 1 - \frac{0.2}{288} {N}_{hist} )$$

to produce a second weighted value. At 1208, the hourly basal insulin needs of the user (i.e., B(k)) is set as the sum of the first weighted value and the second weighted value. The basal delivery history is weighted more as the number of cycles of history increases.

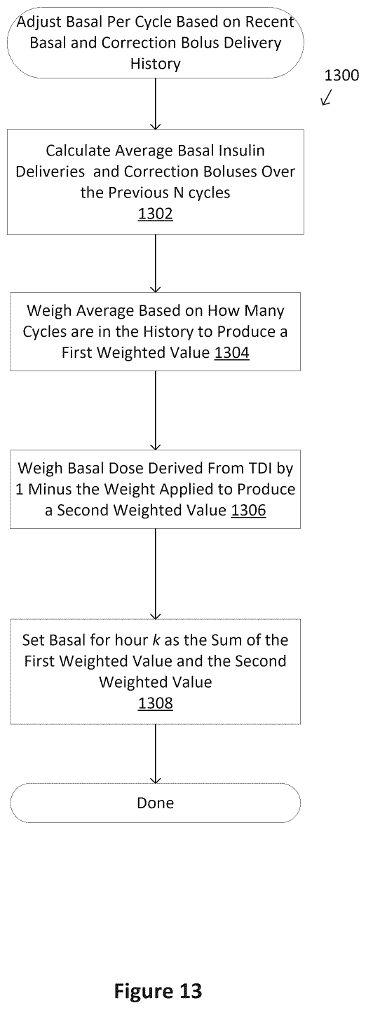

In other exemplary embodiments, the approach of FIG. 12 and Equation 15 may be augmented to calculate the user's basal needs based on the sum of basal deliveries and correction bolus deliveries. FIG. 13 depicts a flowchart 1300 of illustrative steps that may be performed by exemplary embodiments in this augmented approach. The basal needs of the user for hour k may be calculated as:

$$B ( k ) = max \frac{TDI}{48} + min ( 1 , \frac{0.2}{288} {N}_{hist} ) \frac{{\sum }_{j = 1}^{{N}_{hist}} {I}_{basal} ( k - j ) + {I}_{b o l u s , c o r r} ( k - j )}{\frac{{N}_{h i s t}}{288} / 24} \qquad ( Equation 17 )$$

At 1302, the average of the sum of basal insulin deliveries and correction boluses over the past N cycles is calculated

$$( i . e . , \frac{{\sum }_{j = 1}^{{N}_{{hist}_{{I}_{basal ( k - j ) + {I}_{b o lus , corr} ( k - j )}}}}}{\frac{{N}_{hist}}{288} / 24} ) .$$

At 1304, the average is weighed based on how many cycles are in the history to produce a first weighted value. The weight may be min

$$( 1 , \frac{0.2}{288} {N}_{hist} )$$

as discussed above relative to FIG. 12, and the weighted average is the product of the average of the sum calculated in 1302 and the weight. At 1306, the standard basal hourly dose (i.e., TDI/24 divided by 2 or TDI/48) is weighed by

$$max ( 0 , 1 - \frac{0.2}{288} {N}_{hist} )$$

to produce a second weighted value. At 1308, the hourly basal delivery amount for hour k is set as the sum of the first weighted value and the second weighted value.

The exemplary embodiments may attempt to account for inaccuracies in a user's non-bolus insulin needs by removing any basal deviations following user boluses that are paired with significant glucose excursions. In this case, the hourly basal insulin needs of the user for hour k may be calculated as:

$$B ( k ) = max ( 0 , 1 - \frac{0.2}{288} {N}_{hist} ) \frac{TDI}{48} + min ( 1 , \frac{0.2}{288} {N}_{hist} ) \frac{{\sum }_{j = 1}^{{N}_{hist}} {I}_{basal , true} ( k - j )}{\frac{{N}_{hist}}{288} / 24} \qquad ( Equation 18 )$$

$$where$$

$${I}_{b a sal , true} ( k - j ) = \{ \begin{matrix}B ( k - 1 ) / 12 & G ( k - j ) \geq 180 and {k}_{since last bolus} \leq 60 \\ B ( k - 1 ) / 12 & G ( k - j ) \leq 80 and {k}_{since last bolus} \leq 60 \\ {I}_{basal} ( k - j ) & otherwise\end{matrix} . \qquad ( Equation 19 )$$

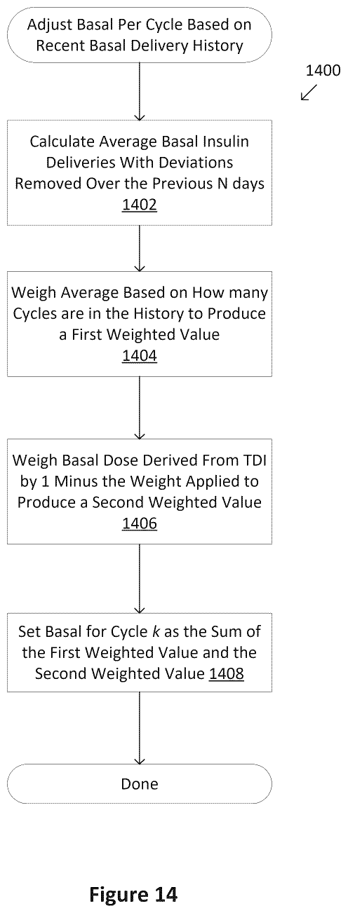

FIG. 14 depicts a flowchart 1400 of illustrative steps that may be performed in exemplary embodiments to determine the hourly basal needs of the user per Equations 18 and 19. At 1402, the average basal insulin deliveries with deviations removed over the previous Nₕᵢₛₜ cycles may be determined. This average is represented in Equation 18 as

$$\frac{{\sum }_{j = 1}^{{N}_{h i s t}} {I}_{basal , true} ( k - j )}{\frac{{N}_{hist}}{288} / 24} .$$

As can be seen in Equation 19, I_{basal,true}(k−j) is set at B(k−1)/12 if the glucose level value for the cycle k−j is greater than or equal to 180 mg/dL or less than or equal to 80 mg/dL and the cycle k−j is within 5 hours of an insulin bolus delivery, the value B(k−1)/12 is used in determining the average. Otherwise, the basal insulin delivery for cycle k−j is used in determining the average. At 1404, the average may be weighed by how many cycles are in the history (specifically by

$$min ( 1 , \frac{0.2}{288} {N}_{hist} )$$

to produce a first weighted value. At 1406, the basal dose derived from TDI

$$( i . e . , \frac{TDI}{48} )$$

is weighed by

$$max ( 0 , 1 - \frac{0.2}{288} {N}_{hist} )$$

to produce a second weighted value.

While exemplary embodiments have been described herein, various changes in form and detail may be made without departing from the claims appended hereto and equivalents thereof.

1. A medicament delivery system, comprising:
a non-transitory computer-readable storage storing computer programming instructions;
a processor configured for executing the computer programming instructions to cause the processor to:
determine a proportionality parameter for a user based on a quantity of insulin that was delivered manually to the user and that is still active over a previous period and/or an amount of insulin on board (IOB) that is still active and that was delivered as one or more meal boluses to the user over the previous period; and
determine basal needs of the user for a time window as a portion of the total daily insulin (TDI) of the user determined by the proportionality parameter.

2. The medicament delivery system of claim 1, wherein the quantity of insulin that was delivered manually to the user and that is still active over the previous period is a quantity of manually delivered correction boluses over the previous period.

3. The medicament delivery system of claim 1, wherein the quantity of insulin that was delivered manually to the user and that is still active over the previous period is a quantity of manually delivered meal and correction boluses over the previous period.

4. The medicament delivery system of claim 1, wherein the previous period is 5 hours in length.

5. The medicament delivery system of claim 1, wherein the determining of the basal needs of the user for a time window comprises multiplying the proportionality parameter by total daily insulin (TDI) divided by 24.

6. The medicament delivery system of claim 1, wherein the time window is an hour in length.

7. The medicament delivery system of claim 1, wherein the proportionality parameter is constrained to assume a value in a range extending from a minimum value to a maximum value.

8. A medicament delivery system, comprising:
a non-transitory computer-readable storage storing computer programming instructions;
a processor configured for executing the computer programming instructions to cause the processor to:
determine a reliability factor for a current total daily insulin (TDI) value for a user based on an amount that a current glucose level value of the user differs from a target glucose level for the user and based on variance of TDI values over time; and
determine basal insulin needs over a time window for a user by applying the reliability factor to the current TDI value.

9. The medicament delivery system of claim 8, wherein the determining of the basal insulin needs comprises multiplying the reliability factor by a tuning factor to yield a product.

10. The medicament delivery device of claim 9, wherein the determining of the basal insulin needs further comprises adding the product with an additional tuning factor to determine a sum and multiplying the current TDI value divided by how many time periods of a length equal to a length of the time window are in a day to determine the basal insulin needs over the time window.

11. The medicament delivery system of claim 9, wherein the determining of the reliability factor comprises determining a standard deviation of the TDI values over a period of multiple days.

12. The medicament delivery system of claim 8, wherein the reliability metric is constrained to have a value greater than zero.

13. The medicament delivery system of claim 8, wherein the time window is an hour in duration.

14. A medicament delivery system that includes a medicament delivery device for delivering insulin to a user, comprising:
a non-transitory computer-readable storage storing computer programming instructions;
a processor configured for executing the computer programming instructions to cause the processor to:
sum basal insulin deliveries by the medicament delivery device to the user and correction bolus deliveries to the user over a period exceeding a day to determine a total, wherein the period contains intervals;
determine an average of the basal insulin deliveries per interval for the total; and
determine an amount of basal insulin to be delivered over a next interval based on the determined average of the basal insulin deliveries per interval.

15. The medicament delivery system of claim 14, wherein the computer programming instructions further cause the processor to identify basal medicament deliveries that are postprandial.

16. The medicament delivery system of claim 15, wherein in summing the basal medicament deliveries, for a non-postprandial basal medicament delivery at a cycle where a glucose reading for the cycle is above a high glucose level threshold, replacing the insulin amount that was delivered at the cycle with an amount equal to an input hourly basal amount of the user divided by a number of cycles in an hour.

17. The medicament delivery system of claim 15, wherein in summing the basal medicament deliveries, for a non-postprandial basal medicament delivery at a cycle where a glucose reading for the cycle is below a low glucose level threshold, replacing the insulin amount that was delivered at the cycle with an amount equal to an input hourly basal amount of the user divided by a number of cycles in an hour.

18. The medicament delivery system of claim 14, wherein the determined average of the basal insulin deliveries per interval is the determined amount of basal to be delivered over the next interval.

19. The medicament delivery system of claim 14, wherein the amount of basal insulin to be delivered of the next interval is a sum of a weighted value derived from total daily insulin (TDI) of the user and a weighted value of the determined average of the basal insulin deliveries per interval.
